(19) United States
(12) Patent Application Publication
(10) Pub. No.: US 2016/0095552 A1
(43) Pub. Date: Apr. 7, 2016

(54) NON-INVASIVE RADIOFREQUENCY COIL FOR MAGNETIC RESONANCE IMAGING

(71) Applicant: The Regents of the University of California, Oakland, CA (US)

(72) Inventors: Seunghoon Ha, Eden Prairie, MN (US); Werner W. Roeck, Irvine, CA (US); Orhan Nalcioglu, Newport Coast, CA (US)

(21) Appl. No.: 14/704,767

(22) Filed: May 5, 2015

Related U.S. Application Data
(60) Provisional application No. 61/988,480, filed on May 5, 2014.

(51) Int. Cl. A61B 5/00; A61B 5/055; G01R 33/34

(52) U.S. Cl. CPC A61B 5/6887; G01R 33/34; A61B 5/055; A61B 5/4381

(57) ABSTRACT

The disclosure relates to a non-invasive, radiofrequency (RF) coil array comprising at least two loop coils configured to wrap in close proximity around a specific anatomical region of a subject, a magnetic Resonance Imaging (MRI) system comprising the RF coil array, and a method of magnetic resonance imaging.

### INCORPORATION BY REFERENCE TO ANY PRIORITY APPLICATIONS

This application claims priority U.S. Provisional Application No. 61/988,480 filed on May 5, 2014. Any and all applications for which a foreign or domestic priority claim is identified in the Application Data Sheet as filed with the present application are hereby incorporated by reference under 37 CFR 1.57.

### BACKGROUND

The disclosure relates to a non-invasive, radiofrequency (RF) coil array, a Magnetic Resonance Imaging (MRI) system and methods of using the MRI system. In magnetic resonance imaging (MRI), the detection sensitivity of a radiofrequency (RF) loop coil dependents on the distance of the organ to the coil, as well as the coil's shape, size, and orientation. The closer the RF coil is placed to the region-of-interest (ROI), the better the signal-to-noise ratio (SNR) of the resulting MR image (Hayes and Axel 1985). Thus instead of using a general-purpose RF coil (e.g. the cylindrical birdcage coil), dramatic improvements in image quality can be achieved by utilizing an array of loop coils configured for the specific anatomy of interest. The loop coils can be designed and positioned to be as close to the ROI as possible to achieve the maximum SNR.

The torso-pelvic receiver array coil is one such example of tailoring the design for a specific anatomy, in this case the overall pelvic region which includes the prostate. However, the internal position of the prostate, and the distance to the external coils limits its detection sensitivity. As such, prostate MRI using only the torso-pelvic coil lacks sufficient SNR to visualize transcapsular tumor spread or seminal vesicle involvement, resulting in a decrease in staging accuracy and specificity (Fütterer et al 2007, Heijmink et al 2007). To address the SNR requirements for prostate MRI, an endorectal coil can be utilized instead. This coil is placed internally in close proximity to the prostate, resulting in a substantial increase in the SNR from the prostate. However due to its limited field-of-view (FOV) and non-uniform sensitivity, the endorectal coil can be used in combination with the torso-pelvic coil to provide full coverage of the pelvic region. This approach provides the high SNR of the prostate along with a large FOV to evaluate the pelvic lymph nodes and pelvic bones for metastatic disease. The combination of endorectal and torso-pelvic coils is the current state-of-the-art approach for prostate MRI (Turkbey et al 2009).

### SUMMARY OF THE INVENTION

Some embodiments relate to a non-invasive, radiofrequency (RF) coil array including a plurality of loop coils configured to wrap in close proximity around a specific anatomical region of a subject.

In some embodiments, the specific anatomical region is the anteroposterior region of the inferior pelvic abdomen and rectum of a subject.

In some embodiments, the plurality of receiver coils comprises a central coil loop and a one or more loop coil(s) positioned adjacent to and on each side of the central loop.

In some embodiments, the RF coil array includes five coils, wherein two pairs coils are positioned adjacent to and on each side of a central coil loop.

In some embodiments, the coils include one or more copper printed circuit board(s) (PCB), wherein circuit patterns for the loop coils are etched on the PCB.

In some embodiments, the RF coil array includes a central, butterfly shaped loop coil.

In some embodiments, non-adjacent coil elements in the plurality of coils are decoupled by low noise amplifiers (LNAs).

Some embodiments relate to a magnetic Resonance Imaging (MRI) system including an RF coil array disclosed herein, wherein the plurality of receiver coils are connected to the MRI system.

In some systems, the RF coil array is connected to the MRI system by one or more coaxial cables.

Some embodiments relate to a method of magnetic resonance imaging including: placing an RF coil array as disclosed herein in close proximity to a specific anatomical region of a subject, and scanning the specific anatomical region of the subject by magnetic resonance imaging.

In some embodiments, the method further includes providing a medical diagnosis pertaining to said subject.

In some embodiments of magnetic resonance imaging, the specific anatomical region of the subject is the anteroposterior region of the inferior pelvic abdomen and rectum of a subject.

Some embodiments of the disclosed method include a step of visualizing a prostate region of said subject.

### BRIEF DESCRIPTION OF THE DRAWINGS

FIG. 1. Side view (a), front view (b) and top view of the diaper coil design
(c), a top view of the constructed pelvic phantom and diaper coil (d), and coil simulation setup with the dielectric phantom (e), (f), (g), (h) and (i) with simulated B1-field sensitivity maps for individual coils. The maps were normalized by 1 W/3.0×10-022 W RF source at 127.7 MHz and the sensitivity range of map was from 0.3 μT to 2.0 μT. The experimental setup for embodiments of the RF coil array are shown (j), the torso-pelvic coil (k), and endorectal coil (1).

FIG. 2. A circuit diagram of the diaper array coil and isolation measurement table between coil elements.

FIG. 3. Sagittal MR images of the phantom using the (a) diaper, (b) torso-pelvic and (c) endorectal coil. The circle areas are indicated as the bladder phantom and the prostate phantom. SNR measurements around the prostate region from axial MR slice using the (d) diaper, (e) torso-pelvic and (f) endorectal coil.

### DETAILED DESCRIPTION

The combined use of a torso-pelvic RF array coil and endorectal RF coil is the current state-of-the-art in prostate MRI. The endorectal coil provides high detection sensitivity to acquire high-spatial resolution images and spectroscopic data, while the torso-pelvic coil provides large coverage to assess pelvic lymph nodes and pelvic bones for metastatic disease. However, the use of an endorectal coil is an invasive procedure that presents difficulties for both patients and technicians. We disclose here a novel, non-invasive RF coil design that provides both image SNR and FOV coverage comparable to the combined torso-pelvic and endorectal coil configuration. A prototype coil was constructed and tested using a pelvic phantom. The results demonstrate that this new design is a viable alternative for prostate MRI.

Despite the improved image quality, the use of an endorectal coil is an invasive, highly uncomfortable procedure that requires a specialized technician to properly position the device and carefully monitor RF heating. Its discomfort for patients and difficulty for technicians poses a significant limitation that impedes the widespread use of prostate MRI. The development of a non-invasive RF coil that provides comparable SNR to the endorectal coil greatly contributes to the full clinical potential of prostate MRI. To this end, a new diaper-shaped array coil (henceforth referred to as the “diaper coil”) was designed and tested for significantly improving the image SNR and uniformity of the prostate region. A prototype coil was constructed on an acrylic frame and evaluated through 3 T MR imaging of a pelvic phantom. Imaging was also performed using a commercial 6-channel torso-pelvic array coil and single-loop endorectal coil for comparison.

### Example 1

### RF Coil Design & Experimental Setup

At first, the proposed RF coil and dielectric phantom were modeled using a full wave electromagnetic field simulation program (SEMCAD X, Ver. 14.2.1; Schmid & Partner Engineering AG, Zurich, Switzerland) to visualize the B1-field maps for individual loop coils. For the dielectric phantom, the dielectric properties of the abdomen (relative permittivity ∈r=69.0, electric conductivity σ=1.50 S/m), bladder (∈r=21.9, σ=0.30), and prostate (∈r=72.2, σ=0.93) were selected to mimic mean tissue values at 127.7 MHz (Gabriel et al 1996).

To eliminate the use of the invasive endorectal coil while still obtaining high SNR within the prostate region and coverage across the pelvis, we developed a new design consisting of a diaper-shaped array of receiver coils placed around the anteroposterior region of the inferior pelvic abdomen and rectum. To test the efficacy of this design concept, we constructed and tested a five-segment prototype as shown in FIG. 1. The bottom, central portion of the array consists of a butterfly-shaped loop coil (width=80 mm, length=150 mm) orientated parallel to the x-y plane such that it generates a B1-field along the y-axis. A pair of loop coils (width=130 mm, length=80 mm) inclined 12 and 30 degrees about the x-axis were positioned on opposite sides of central portion of the array to form the diaper shape. The coils were made from copper printer circuit board (PCB) traces (width=5 mm, thickness=34 μm) and positioned with optimum overlap to minimize mutual coupling between adjacent elements (Roemer et al 1990).

The performance of this diaper coil was compared to a commercial 6-channel torso-pelvic array coil (USA Instruments, Inc.; Aurora, Ohio) and endorectal coil (Medrad, Inc.; Indianola, Pa.) though the imaging of an in-house-built pelvic phantom. The torso-pelvic coil consists of 6 non-overlapping rectangular loops (width=125 mm, length=235 mm). Three loops are placed above the patient, while 3 loops are positioned below the patient, providing coverage of 395 mm along the x-axis and 180 mm along the y-axis. The endorectal coil consists of a single rectangular loop (width=30 mm, height=80 mm) formed by a thin copper strip (width=2 mm, thickness=34 μm). The pelvic phantom was constructed using acrylic for the oval cylinder (width=300 mm, length=300 mm, height=190 mm) to mimic the abdomen and hollow polypropylene balls for the bladder and prostate regions. A hollow polypropylene ball to model the bladder (diameter=100 mm) and a smaller hollow polypropylene ball to model the prostate (diameter=35 mm) were positioned within the cylinder to mimic human anatomy (Schulte et al 2006, Standring et al 2008, Yokochi et al 1978).

To mimic the dielectric properties of the human body, we followed the approach of Yang et al (2004), where the concentration of NaCl in solution relative to the phantom volume was adjusted so that the ratio σ/ω∈0∈r was the same as in human tissue, where w is the angular (Larmor) frequency and ∈0 is the permittivity of free space. The phantom was filled with 120 mM, 20 mM, and 70 mM of NaCl solution in the abdomen, bladder and prostate regions respectively. 10 mM of CuSO4 was also added to each region to improve image quality in T1-weighted imaging.

The diaper coil was positioned across the curved face of phantom. The torso-pelvic coil was wrapped on the top and bottom faces of the phantom. The endorectal coil was positioned within the phantom directly underneath the sphere representing the prostate.

### Example 2

### Coil Construction and MRI Experiments

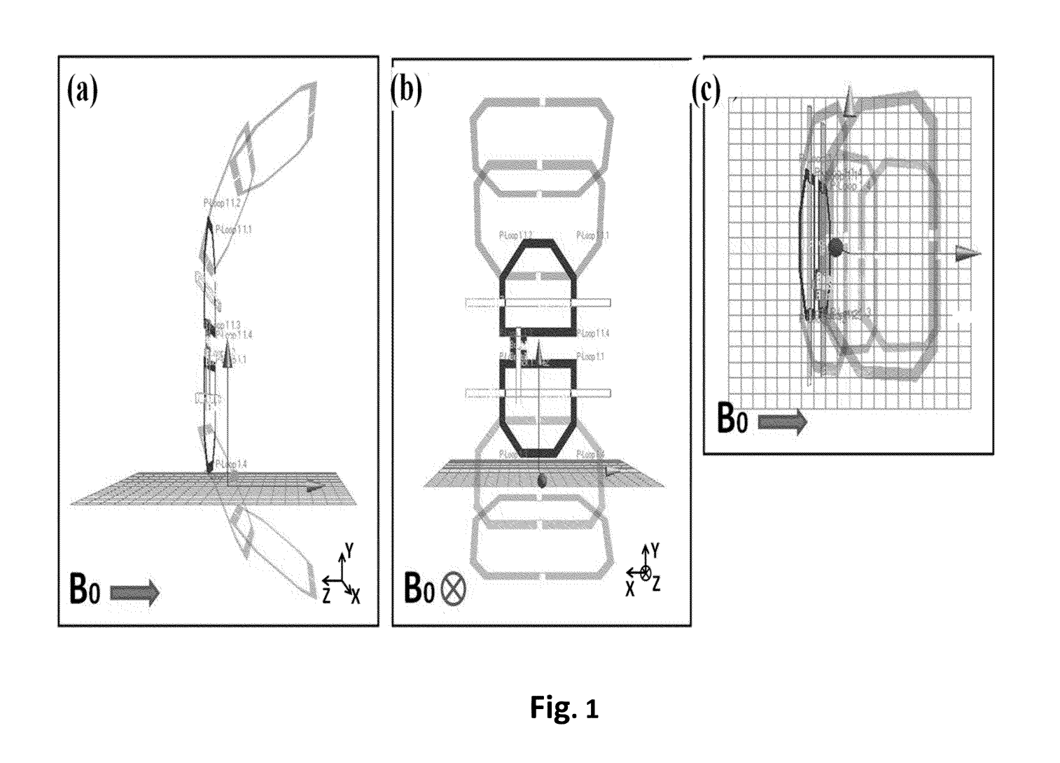

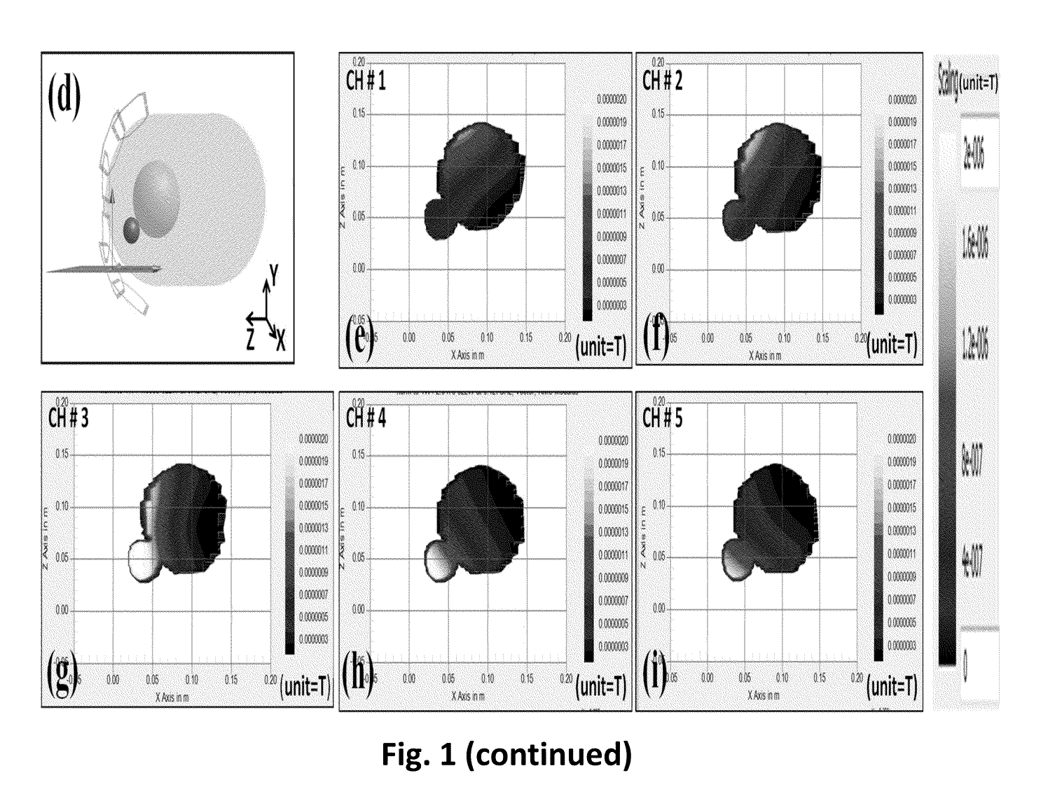

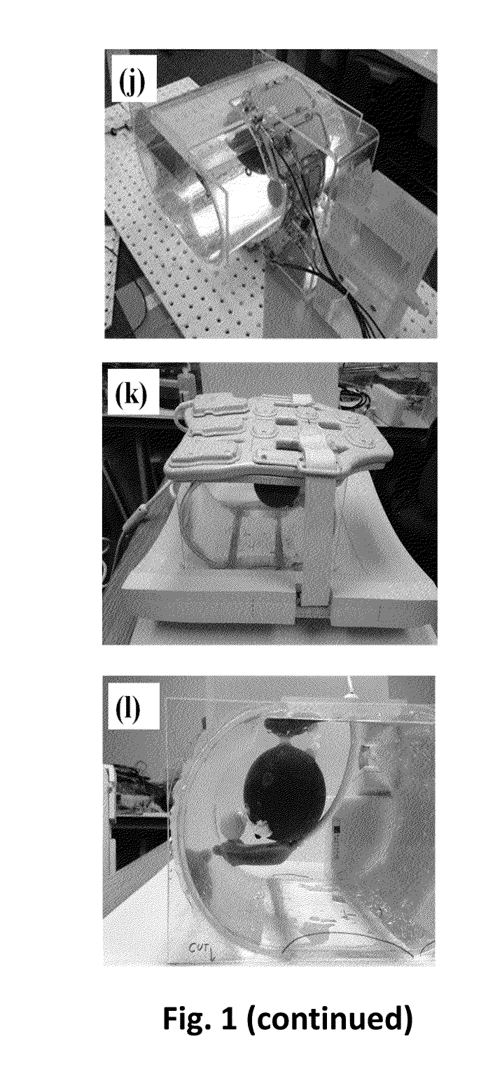

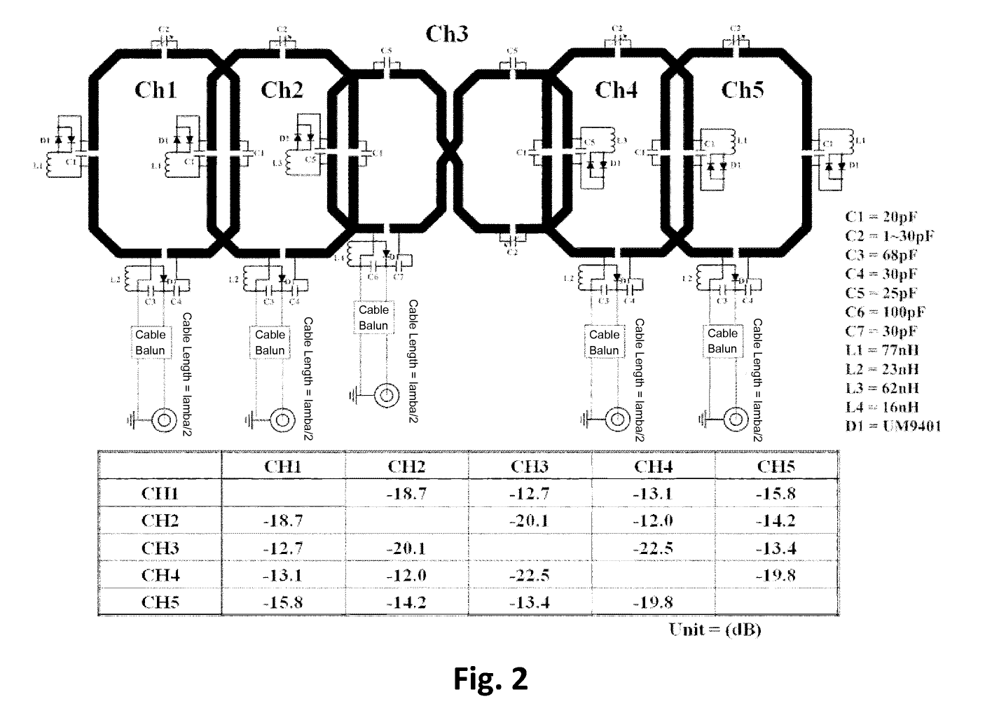

For constructing the diaper array coil, the circuit pattern for each loop coil was etched on a separate printed circuit board (PCB) composed of flame-retardant G-10 plastic. Discrete capacitors (ATC 100B Series Porcelain Superchip Multilayer Capacitor; American Technical Ceramics Corporation, Huntington Station, USA) and a trimmer capacitor (NMAF30; Voltronics Corporation, Denville, USA) were soldered onto each PCB as shown in FIG. 2. The individual coils were tuned to 127.7 MHz and the impedance matched to 50Ω (with phantom loading). Since the array operates in receive-only mode, passive and active detuning circuits containing PIN diodes (UMX9989AP (passive detuning) UM9401 (active detuning); Microsemi, USA) were integrated into the circuit pattern to decouple the coils from the RF transmitter during high power RF transmission. Isolation between adjacent coil elements was measured from the S21 parameters after plugging the two coils to a network analyzer (4395A; Agilent Technologies, USA). After the coil elements were mounted on an acrylic half-cylinder to form the diaper shape, the optimum coil overlap between adjacent coils to reduce mutual coupling was achieved when the isolation measured below −17 dB. The coil elements among non-adjacent coil elements were also decoupled by low noise amplifiers (LNAs: Philips Medical Systems, Netherlands) mounted in a coil interface box (Philips Medical Systems, Netherlands).

To interface the array coil with the MRI scanner, we prepared five coaxial cable (length=λ/2) and connector assemblies each containing a cable balun tuned to 127.7 MHz. For each assembly, one cable end was soldered to the capacitive matching circuit of one coil element and the other end was connected to the coil interface box. Eight LNAs with 25 dB gain, 0.4 dB noise figure, and 5±1Ω input impedance tuned to 127.7 MHz were also mounted in the coil interface box. Identical LNAs were employed for the torso-pelvic and endorectal coils. The low input impedance of the LNAs effectively work in conjunction with an individual coil's matching/decoupling circuit to eliminate residual magnetic fields induced in neighboring coil elements, thus further reducing mutual coupling. The coil interface box also provided the voltages (−5V) and currents (150 mA) that drive the PIN diode in each active decoupling circuit.

MR images of the phantom were acquired using all three coils (diaper, torso-pelvic, endorectal) with a 3 T Philips Achieva system (Philips Medical Systems) with the following scan parameters: sequence=T1-weighted gradient echo (T1W-FFE), repetition time (TR)=161 ms, echo time (TE)=4.6 ms, flip angle=80°, matrix=528×528, FOV=300 mm×300 mm, slice thickness=3 mm and number of excitations (NEX)=2. Acquired individual coil loop images were combined by most commonly used sum-of-squares algorithm (Roemer et al 1990). The axial slice covering the middle of the prostate region was used to calculate the integral uniformity (IU) and the SNR within a local FOV. The SNR of the local FOV was computed by Eq. [1]. The mean signal (Savg.) was defined as the mean pixel intensity value in a 50 mm ROI covering the prostate phantom. The mean noise (Navg.) as well as the standard deviation (σ) is measured on the image background.

SNR=(S_{avg}−N_{avg})/σ Eq. [1]

The percentage integral uniformity (IU) was calculated as Eq. [2] where the maximum and minimum values were taken from pixels within the prostate region.

$$I U = 100 \times [ \frac{{SNR}_{max} - {SNR}_{min}}{{SNR}_{max} + {SNR}_{min}} ] \qquad Eq . [ 2 ]$$

### Results

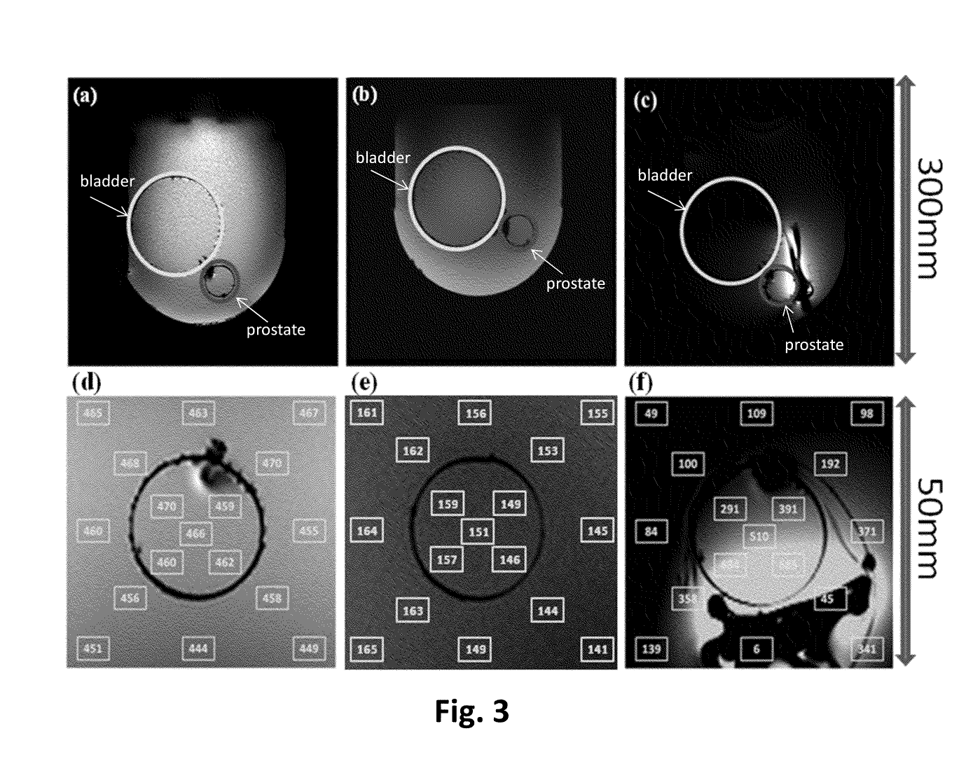

MR imaging of the prostate phantom, shown in FIGS. 3 (a-f), yielded interesting findings. The measured IU results are given in table 1. It should be emphasized that for a given RF coil, the higher the IU, the worse its uniformity. Both the diaper and torso-pelvic coils provided coverage across the whole phantom while the endorectal coil was only effective within a very limited FOV. The detection sensitivity in the prostate region was most homogeneous for the diaper coil, followed closely by the torso-pelvic coil, and highly non-uniform for the endorectal coil. In addition, the diaper coil provided significantly higher image SNR than the torso-pelvic coil. While the (non-uniform) SNR of the endorectal coil within the prostate region ranged from 150 to 710, its mean value was comparable to that of the diaper coil (within 8%).

TABLE 1
Integral Uniformity (IU) of the prostate region
using the three different RF coils.

| | IU (%) |
|---|---|
| Diaper coil | 1.2 |
| Torso-pelvic coil | 7.8 |
| Endorectal coil | 40.4 |

Our results demonstrate that the diaper coil can serve as a noninvasive alternative to the standard torso-pelvic and endorectal coil combination currently used in clinical prostate MRI by providing both comparable image SNR and FOV coverage. While the mean image SNR using the diaper and endorectal coils are similar, the endorectal coil provides superior detection sensitivity in regions closest to the coil.

Our original 5-channel prototype was designed with the concept of positioning the coil between a patient's legs immediately adjacent to the crotch. The shape of the coil was formed with this configuration in mind and tested using phantoms. The ergonomics of the coil design are relevant to in vivo studies. Advantageously, the coil is designed to fit as comfortable as possible, while maintaining its performance quality. Specifically, in some embodiments, edges and sharp bends are shaped to smooth contours, particularly around the patent's crotch. In some embodiments, electronic components are fully insulated from contact with the patient. The rigid cylindrical acrylic shell used to mount the coil loops can be replaced with flexible, folding elements that can form to each patient's unique body shape.

We disclose a new RF coil design (e.g., for prostate MRI, consisting of a diaper-shaped array of receiver (or loop) coils placed around the anteroposterior region of the inferior pelvic abdomen and rectum). The placement of the new RF coil array may be adjacent to, near or in close proximity to (e.g., at a distance no greater than that which provides adequate imaging and no less than non-invasive contact with the subject), around (e.g., at least partially surrounding) the anatomical region of interest. MRI experiments using a phantom demonstrated that the non-invasive diaper coil can provide image SNR and FOV coverage comparable to the torso-pelvic and invasive endorectal coil combination currently used in clinical practice. These results provide a basis for imaging on human subjects.

In one embodiment, after placing the RF coil array adjacent to a specific anatomical region of a subject, and scanning the specific anatomical region of the subject by magnetic resonance imaging, an image is visualized by conventional equipment/methods, and optionally, a medical diagnosis is rendered based on the visualized image.

### REFERENCES

1. Fütterer J J, Engelbrecht M R, Jager G J, Hartman R P, King B F, Hulsbergen-Van de Kaa C A, Witjes J A and Barentsz J O 2007 Prostate cancer: comparison of local staging accuracy of pelvic phased-array coil alone versus integrated endorectal-pelvic phased-array coils. Local staging accuracy of prostate cancer using endorectal coil MR imaging Eur. Radiol. 17 1055-65.
2. Gabriel S, Lau R W, and Gabriel C 1996 The dielectric properties of biological tissue: III. Parametric models for the dielectric spectrum of tissues 1996 Phys. Med. Biol. 41 2271-93.
3. Hayes C E and Axel L 1985 Noise performance of surface coils for magnetic resonance imaging at 1.5 T Med. Phys. 12 604-7.
4. Heijmink S W, Fütterer J J, Hambrock T, Takahashi S, Scheenen T W, Huisman H J, Hulsbergen-Van de Kaa C A, Knipscheer B C, Kiemeney L A, Witjes J A and Barentsz J O 2007 Prostate cancer: body-array versus endorectal coil MR imaging at 3 T—comparison of image quality, localization, and staging performance Radiology 244 184-95.
5. Hoult D I, Chen C N and Snak V J 1984 Quadrature detection in the laboratory frame Magn. Reson. Med. 1 339-53.
6. Roemer P B, Edelstein W A, Hayes C E, Souza S P and Mueller O M 1990 The NMR phased array Magn. Reson. Med. 16 192-255.
7. Schulte R F, Vogel M W, Schirmer T, Schilling H, Groeger A, and Gross P 2006 Design of an anatomically and physiologically realistic prostate phantom. Proc. Intl. Soc. Mag. Reson. Med. 2252.
8. Standring S, Borley N R, Collins P, Crossman A R, Gatzoulis M A, Healy J C, Johnson D, Mahadevan V, Newell R L M, and Wigley C B 2008 Gray's anatomy—The anatomical basis of clinical practice. 4th ed. Spain, Churchill Livingstone 1255-70.
9. Turkbey B, Albert P S, Kurdziel K and Choyke P L 2009 Imaging localized prostate cancer: current approaches and new developments AJR Am. J. Roentgenol. 192 1471-80.
10. Yang Q X, Wang J, Collins C M, Smith M B, Zhang X, Ugurbil K and Chen W 2004 Phantom design method for high-field MRI human systems Magn. Reson. Med. 52 1016-20.
11. Yokochi C and Rohen J W 1978 Photographic anatomy of the human body. 2nd ed. Baltimore, University Park Press 51-54.

The above references are hereby incorporated in their entireties by reference thereto.

What is claimed is:

1. A non-invasive, radiofrequency (RF) coil array, comprising at least two loop coils configured to wrap in close proximity around a specific anatomical region of a subject.

2. The RF coil array of claim 1, wherein the specific anatomical region is the anteroposterior region of the inferior pelvic abdomen and rectum of a subject.

3. The RF coil array of claim 1, wherein the at least two loop coils comprises a central coil loop and one or more loop coil(s) positioned adjacent to and on each side of the central loop.

4. The RF coil array of claim 1, wherein the at least two loop coils comprises five coils, comprising two pairs of coils positioned adjacent to and on either side of a central coil loop.

5. The RF coil array of claim 1, further comprising one or more copper printed circuit board(s) (PCB), wherein circuit patterns for the loop coils are etched on the PCB.

6. The RF coil array of claim 1, wherein the at least two loop coils comprise a central coil loop having a butterfly shape.

7. The RF coil array of claim 1, wherein non-adjacent coil elements in the at least two loop coils are decoupled by low noise amplifiers (LNAs).

8. A magnetic Resonance Imaging (MRI) system, comprising the RF coil array of claim 1, wherein the at least two loop coils are connected to the MRI system.

9. The MRI system of claim 8, wherein the RF coil array is connected to the MRI system by one or more coaxial cables.

10. A method of magnetic resonance imaging, the method comprising:
placing the RF coil array of claim 1 in close proximity to a specific anatomical region of a subject, and
scanning the specific anatomical region of the subject by magnetic resonance imaging.

11. The method of claim 10, wherein the specific anatomical region of the subject is the anteroposterior region of the inferior pelvic abdomen and rectum of a subject.

12. The method of claim 12, further comprising a step of visualizing a prostate region of said subject.

13. The method of claim 10, further comprising providing a medical diagnosis pertaining to said subject.
